(19) United States
(12) Patent Application Publication
(10) Pub. No.: US 2007/0065409 A1
(43) Pub. Date: Mar. 22, 2007

(54) Use of pleiotrophin in the diagnosis, treatment and prevention of disease

(76) Inventors: Behrooz Sharifi, Woodland Hills, CA (US); Prediman K. Shah, Los Angeles, CA (US)

Correspondence Address: DAVIS WRIGHT TREMAINE LLP, 865 FIGUEROA STREET, SUITE 2400, LOS ANGELES, CA, 90017-2566, US

(21) Appl. No.: 10/564,009

(22) PCT Filed: Jul. 15, 2004
(86) PCT No.: PCT/US04/22827

§ 371 Date: Jul. 14, 2006

Related U.S. Application Data
(60) Provisional application No. 60/487,409, filed on Jul. 15, 2003.

(51) Int. Cl. A61K 48/00; C12N 5/08; C12N 15/867

(52) U.S. Cl. 424/93.2; 424/93.7; 435/366; 435/456

(57) ABSTRACT

Described herein are methods for transdifferentiating monocytes/macrophages into endothelial cells, based on the inventors' identification of pleiotrophin (“PTN”) as the gene responsible for this transdifferentiation. Further described are therapeutic methodologies for the promotion or inhibition of neovasculanization based on modulation of PTN.

### FIELD OF THE INVENTION

The invention relates to the diagnosis, treatment and prevention of diseases in which neovascularization is a component of disease pathology as well as diseases in which the promotion of neovascularization is desirable. More specifically, the invention relates to neovascularization related to the effects of pleiotrophin.

### BACKGROUND OF THE INVENTION

The recent explosion in knowledge regarding identification and characterization of physiological regulators of blood vessel growth indicates the need for re-evaluation of therapeutic efforts aimed at regulating blood vessel growth—whether that involves promoting new vascularization to replenish ischemic tissue, blocking vessel growth in order to blunt tumor growth, or repairing damaged and leaky vessels during inflammation or other pathological settings. Monocytes/macrophages play a pivotal role in the development of neovessels in inflamed tissues and tumors. Persistent elevation of monocytes and their conversion to resident macrophages are hallmarks of chronic inflammation. This accumulation results from either increased recruitment of circulating monocytes or proliferation of resident monocytes/macrophages. Recent studies have shown that pleiotrophin (“PTN”) stimulates proliferation of peripheral blood-derived monocytes and tissue macrophages. See, e.g., A. Achour et al., “The angiogenic factor heparin affin regulatory peptide (HARP) induces proliferation of human peripheral blood mononuclear cells,” Cell Mol. Biol. (Noisy-le-grand) 47 Online Pub, OL73-7 (2001); T. Pufe et al., “Expression of pleiotrophin, an embryonic growth and differentiation factor, in rheumatoid arthritis,” Arthritis Rheum 48, 660-7 (2003). PTN is a secreted growth factor that is highly expressed in several primary human tumors and is considered as a rate-limiting angiogenic factor in tumor growth, invasion, and metastasis. T. Muramatsu, “Midkine and pleiotrophin: two related proteins involved in development, survival, inflammation and tumorigenesis,” J. Biochem (Tokyo) 132, 359-71 (2002). The mechanism by which PTN promotes tumor vascularization is unknown.

Monocytes/macrophages are believed to promote neovascularization by releasing a myriad of angiogenic factors and cytokines. Endothelial cells are recruited by angiogenic factors from two sources: the migration and proliferation of pre-existing endothelial cells (angiogenesis), and the recruitment of circulating endothelial progenitor cells. These latter cells can be isolated by primary enrichment of CD34± cells followed by adherence separation on fibronectin-coated tissue culture dishes. See, e.g., T. Asahara et al., “Isolation of putative progenitor endothelial cells for angiogenesis,” Science 275, 964-7 (1997); C. Kalka et al., “Transplantation of ex vivo expanded endothelial progenitor cells for therapeutic neovascularization,” Proc Natl Acad Sci USA 97, 3422-7 (2000); and M. Nieda et al., “Endothelial cell precursors are normal components of human umbilical cord blood,” Br. J. Haematol. 98, 775-7 (1997). In such studies, human peripheral blood mononuclear cells are used as the starting population. These preparations, however, contain varying numbers of monocytes that have high capacity to adhere to extracellular matrix. Thus, it is possible that monocytes also could contribute to the neovascularization attributed to endothelial progenitor cells.

In support of this possibility, several groups have reported that in the presence of angiogenic factors, monocytes isolated from human peripheral blood can transdifferentiate into endothelial-like cells. See, e.g., A. Schmeisser et al., “Monocytes coexpress endothelial and macrophagocytic lineage markers and form cord-like structures in Matrigel under angiogenic conditions,” Cardiovasc. Res. 49, 671-80 (2001); B. Fernandez Pujol et al., “Endothelial-like cells derived from human CD14 positive monocytes,” Differentiation 65, 287-300 (2000); J. Rehman et al., “Peripheral blood ‘endothelial progenitor cells’ are derived from monocyte/macrophages and secrete angiogenic growth factors,” Circulation 107, 1164-9 (2003); and C. Urbich et al., “Relevance of monocytic features for neovascularization capacity of circulating endothelial progenitor cells,” Circulation 108, 2511-6 (2003). Such primary cell preparations, however, contain contaminating cells with the ability to differentiate into endothelial cells. See Y. Zhao et al., “A human peripheral blood monocyte-derived subset acts as pluripotent stem cells,” Proc Natl Acad Sci USA 100, 2426-31 (2003). The issue is further confounded by the ability of monocytes to adopt the phenotype of other cell types by spontaneously fusing with them during cultivation. See N. Terada et al., “Bone marrow cells adopt the phenotype of other cells by spontaneous cell fusion,” Nature 416, 542-5 (2002). In addition, a recent study has shown that circulating cells derived from transplanted bone marrow can fuse with cardiomyocytes, Purkinje cells, and hepatocytes in vivo, raising concern about the validity of transdifferentiation studies involving bone marrow-derived cells. See M. Alvarez-Dolado et al., “Fusion of bone-marrow-derived cells with Purkinje neurons, cardiomyocytes and hepatocytes,” Nature 425, 968-73 (2003). Because a single hematopoietic stem cell is capable of complete repopulation of hematopoietic tissue in lethally irradiated mice, preparations containing even a few such cell contaminants, can in theory confound interpretation of data obtained in transdifferentiation studies. See A. J. Wagers et al., “Little evidence for developmental plasticity of adult hematopoietic stem cells,” Science 297, 2256-9 (2002).

### BRIEF DESCRIPTION OF THE FIGURES

FIG. 1 depicts down-regulation of monocytic cell markers by endogenously produced PTN in accordance with one embodiment of the present invention. FIG. 1A illustrates a semi-quantitative reverse transcriptase-polymerase chain reaction (“RT-PCR”) analysis of monocytic cell markers by THP-1 and endothelial cells. Total RNA was extracted from THP-1 cells grown in 10% serum (lane 2), induced to differentiate into macrophage-like cells by addition of 25 ng/ml PMA (lane 3), transduced with retroviral bicistronic vector expressing: green fluorescent protein (“GFP”) (lane 4), PTN sense strand (lane 5), PTN anti-sense strand (lane 6) followed by treatment with PMA. The exponentially growing human coronary artery endothelial cells (lane 7) were used as a negative control. Analyzed monocytic cell markers were c-fms and CD-68 with primers predicted to amplify 97- and 132-bp DNA fragments, respectively. Glyceraldehyde-3-phosphate dehydrogenase (“GAPDH”) primers were used as control a for the RT-PCR reaction. Lane 1 is a DNA ladder marker. FIG. 1B illustrates expression of CD14 by the monocytic cells. Flow cytometry analysis was performed by incubating 5×10⁵ THP-1 cells expressing PTN (PTN) or GFP (GFP) with PE-labeled anti-CD14 antibody (obtained from PharMingen). Human coronary artery endothelial cells were used as a negative control. Uninfected THP-1 cells (THP-1) were used as positive control and human coronary endothelial cells (endothelial) were used as a negative control. Fluorescence-activated cell sorting (“FACS”) analysis was performed at the Cedars-Sinai Research Institute Core Facility. Each experiment was repeated three times and each bar graph represents mean±SEM of three experiments. FIG. 1C illustrates an amplification plot for CD68 expression in the monocytic cells. Oligonucleotide primer pairs for CD68 gene and an oligonucleotide probe labeled with a reporter fluorescent dye at the 5′ end and quencher fluorescent dye at the 3' end were designed using Oligo 4.0 software (available from National Bioscience, Plymouth, Minn.). Total RNA with DNase I treatment was used to synthesize first-stand cDNA with RT (obtained from GIBCO-BRL) and oligo(dT) 15 Primer (obtained from Promega). Total RNA (50 ng) was added to a 50 μl RT-PCR reaction mixture according to the manufacturer's protocol (Roche Molecular Systems). The products of the RT reactions were used to seed real-time PCR by using an ABI Prism 7700 Sequence Detector by comparing with glyceraldehyde-3-phosphate dehydrogenase (internal control) and individual standard curve with three time repeats. The thermal cycling conditions included one cycle at 48° C. for 30 minutes, one cycle at 95° C. for 10 minutes, 40 cycles at 95° C. for 15 s, annealing at 60° C. for 1 minute, and a final hold at 25° C. for 2 minutes. Standard curves for the expression of each gene were generated by serial dilution of a standard preparation of total RNA isolated from cultured monocytic or endothelial cells.

FIG. 2 depicts expression of endothelial cell markers in monocytic cells by endogenously produced PTN, in accordance with an embodiment of the present invention. FIG. 2A illustrates a semi-quantitative PCR analysis of endothelial cell markers. RNA was isolated and separated by agarose gel electrophoresis. Mouse RAW (lane 1), human THP-1 (lane 2), and human U937 (lane 3) monocytic cells were tested. Non-monocytic cells, NIH 3T3 cells (lane 4), human coronary artery smooth muscle cells (lane 5), RPMI 8226 B lymphocyte plasmacytoma cell line (lane 7), and human dermal fibroblasts (lane 8), were used as negative controls. Human coronary artery endothelial cells (lane 6, obtained from Cell Applications, Inc.) were used as a positive control. In addition, RNA was isolated from THP-1 cells transduced with a bicistronic retroviral vector harboring PTN sense strand (lane 9), PTN anti-sense strand (lane 10), or GFP (lane 11). The RNA was subjected to RT-PCR analysis, using specific primers for vascular endothelial growth factor receptor-2 (Flk-1) [See T. P. Yamaguchi et al., “flk-1, an flt-related receptor tyrosine kinase is an early marker for endothelial cell precursors,” Development 118, 489-98 (1993)], tyrosine kinase receptor Tie-2 [See T. N. Sato et al., “Tie-1 and tie-2 define another class of putative receptor tyrosine kinase genes expressed in early embryonic vascular system,” Proc. Natl. Acad. Sci. USA 90, 9355-8 (1993)], vascular endothelial-cadherin (VE-cad) [See M. G. Lampugnani et al., “A novel endothelial-specific membrane protein is a marker of cell-cell contacts,” J. Cell. Biol. 118, 1511-22 (1992)], PECAM-1 [See H. M. DeLisser et al., “Molecular and functional aspects of PECAM-1/CD31,” Immunol. Today 15, 490-5 (1994)], endothelial nitric oxide synthase (eNOS) [See S. Moncada et al., “Nitric oxide: physiology, pathophysiology, and pharmacology,” Pharmacol. Rev. 43, 109-42 (1991)], the von Willebrand factor (vWf) [Id.], CD34 [See P. E. Young et al., “The sialomucin CD34 is expressed ON HEMATOPOIETIC CELLS AND blood vessels during murine development,” Blood 85, 96-105 (1995)], and AC133/CD133, a cell surface marker of vascular/hematopoietic stem and progenitor cells [See M. Peichev et al., “Expression of VEGFR-2 and AC133 by circulating human CD34(+) cells identifies a population of functional endothelial precursors,” Blood 95, 952-8 (2000)]. To ensure semi-quantitative results of the RT-PCR analysis, the number of PCR cycles for each set of primers was checked to be in the linear range of the amplification. In addition, all RNA samples were adjusted to yield equal amplification of GAPDH as an internal standard. The amplified products were separated on 1.2% agarose gels and stained with ethidium bromide. FIG. 2B illustrates an amplification plot for the expression of VE-cadherin, vWf, and PECAM-1 in the monocytic cells. Total RNA was prepared as described above. Probes were labeled with quencher and fluorescent dye 6-FAM by 3′ and 5′ ends, respectively. cDNA reverse-transcribed from three different preparations of RNA are illustrated; it was isolated from: THP-1 cells infected with PTN sense strand (blue, dark blue, magenta), human coronary artery endothelial cells (dark green, deep blue, and light red), and THP-1 cells infected with retrovirus harboring the GFP gene (yellow, light green, and dark red). FIG. 2C illustrates a spatial distribution of endothelial cell markers in the monocytic cells. GFP-expressing (top panel) or PTN-expressing THP-1 cells (lower panel) were cultured on cover slips in the presence of 10% (vol/vol) FBS followed by addition of PMA as described above. Cells were fixed with methanol for 5 min at 20° C. or with 3% (wt/vol) paraformaldehyde at room temperature and stained for 30 min with anti-human FLK-1 mouse monoclonal (obtained from Santa Cruz Biotechnology, Inc.). The secondary antibody was HRP-labeled goat anti-mouse antibody (obtained from Santa-Cruz Biotechnology, Inc.). The antibodies were used at the dilutions recommended by the manufacturer. FIG. 2D illustrates flow cytometry analysis of Tie-2 expression in the monocytic cells. 5×10⁵ GFP-expressing (top panel) or PTN expressing (lower panel) THP-1 cells were incubated with a 1:100 dilution of anti-human Tie-2 rabbit polyclonal antibody (obtained from Santa Cruz Biotechnology, Inc.) for 30 min on ice. After washing, cells were incubated with 1:500 dilution of PE-labeled anti-rabbit antibody (obtained from Molecular Probes) for 15 min on ice followed by fixing cells with 1% paraformaldehyde. FACS analysis was performed as described above. The figure shows a representative experiment that was repeated with three different PTN-expressing THP-1 cells. Tick marks on the x-axis designate logarithmic increments in fluorescent intensity. FIG. 2E issultrates flow cytometry analysis of α_{v 3}integrin expression by the monocytic cells. GFP-expressing (top left panel) or PTN-expressing THP-1 cells (lower left panel) were incubated with a 1:100 dilution of anti-human α_{v 3}mouse antibody (obtained from Chemicon Co). After washing, cells were incubated with 1:500 dilution of PE labeled anti-mouse antibody (obtained from Sigma), fixed and analyzed by FACS, as described above. In addition, human coronary artery endothelial cells in the absence (top right panel) or presence of anti-α_{v 3}antibody (lower right panel) were used as a positive control.

FIG. 3 depicts transcriptional regulation of transdifferentiation of monocytes by endogenously produced PTN, in accordance with an embodiment of the present invention. FIG. 3A illustrates semi-quantitative RT-PCR for the expression of GATA-2 and GATA-3 in the monocytic cells. Total RNA isolated from cells was subjected to semi-quantitative RT-PCR analysis as described above using primers specific for GATA-2, GATA-3, and Oct-4 transcription factors. The amplified PCR products were analyzed by agarose gel electrophoresis. FIG. 3B illustrates confocal microscopy, for which the secondary antibodies (Alexa Fluor 633 goat anti-rabbit antibody (blue) and Alexa Fluor 568 goat anti-mouse antibody (red)) were used at 1:500 dilutions, as recommended by the manufacturer (Molecular Probes). After the indirect immunolabeling, cells were mounted in Floromount-G (obtained from Southern Biotechnology) and examined with either a conventional fluorescence microscope (obtained from Nikon) or a Zeiss LSM 510 confocal microscope.

FIG. 4 depicts in vitro activity of transdifferentiated monocytic cells, in accordance with an embodiment of the present invention. Fibrin gels were prepared essentially as described in P. Koolwijk et al., “Cooperative effect of TNFalpha, bFGF, and VEGF on the formation of tubular structures of human microvascular endothelial cells in a fibrin matrix. Role of urokinase activity,” J. Cell. Biol. 132, 1177-88 (1996). GFP-expressing or PTN-expressing RAW cells (5×10⁵) were seeded on the fibrin matrix, incubated at 37° C. for 18 days, and the formation of tubular structures was analyzed by phase-contrast microscopy.

FIG. 5 depicts in vivo functional activity of transdifferentiated monocytic cells in quail chorioallantotic membrane (“CAM”) studies, in accordance with an embodiment of the present invention. Fertilized eggs of Japanese quail (Coturnix coturnix japonica, obtained from Boyd's Bird Co., Pullman, Wash.) were incubated at 37° C. under ambient atmosphere, for 56 hours, and then opened at embryonic day 3 (E3) after incubation of the eggs and cultured further at 37° C. in 6-well plates. At E7 (FIG. 5A), 1×10⁶ RAW or 293 cells in prewarmed PBS (200 μl) was applied in drops to the surface of each CAM. The embryos were incubated further at 37° C. for 72 hours (FIG. 5B), at which time they were fixed in 4% paraformaldehyde/2% glutaraldehyde/PBS. Fluorescent and confocal images of terminal arterial vessels from the middle region of the CAM were acquired in gray scale at a total magnification of 2× (FIG. 5C), 10× (FIG. 5D), and 20× (FIG. 5E) and resolution of 13 μm per pixel. There was no significant increase in vessel density at a total magnification of 20× as compared with 10×. A total of 60 CAM specimens were used; 12 CAMs/group. Five independent experiments were performed: RAW cells expressing PTN or THP-1 cells expressing PTN as treatment groups. RAW cells expressing GFP (FIG. 5F) were used as control groups. The fixed CAMs with fluorescence images overlapped with DIC (FIGS. 5G and 5H, respectively) images at 4× magnification.

FIG. 6 depicts in vivo functional activity of transdifferentiated monocytic cells in chicken mouse chimera, in accordance with an embodiment of the present invention. Mouse monocytic RAW cells (1-2×10⁵ cells in 2-4 μl PBS) expressing PTN and GFP were injected into the hearts of stage 16-17 chick embryos. Embryos were killed 2-3 days after injection, fixed, embedded in OTC, and frozen sections were cut and stained with anti-GFP polyclonal antibody (obtained from Santa Cruz Biotechnology, Inc.). The immunopositivity (brown color) was observed in embryo injected with RAW cells expressing PTN/GFP. The positivity localized along the developing blood vessels in the head, eyes, and intersomitic region (right panel). In contrast, immunostaining of embryo injected with RAW cells expressing the GFP gene showed no staining (left panel). In some cases, faint staining was detected around the amniotic cavity.

FIG. 7 illustrates hindlimb ischemia, in accordance with an embodiment of the present invention. BALB/C male mice (obtained from Jackson Laboratories; 12-week-old) were sedated and a longitudinal incision was made on the medial side of the thigh inferior to the inguinal ligament to a point proximal of the patella. The right femoral artery was ligated with 6-0 silk sutures at its proximal origin from the iliac artery and distally at the bifurcation into the popliteal and saphenous arteries in order to induce mild ischemia. RAW cells expressing PTN/GFP (1×10⁶ cells in 200 μl PBS/mouse) were injected intravenously (tail vein) 24 h after surgery (8 mice/group). Mice injected with 200 μl of PBS were used as controls (8 mice/group). Repeated hindlimb blood flow measurements over the region of interest (from the patella to the midfoot) were obtained at baseline, immediately after surgery, and serially over 3 weeks by Laser Doppler Perfusion Imaging (obtained from Moor Instruments) (FIG. 7A). Perfusion is expressed as a ratio of right (ischemic) to left (normal) limb. Representative color-coded images (red is highest velocity, green intermediate, and blue, lowest velocity) that reflect red blood cell velocity at day 7 post surgery are shown. The top 4 panels show BALB/C mice injected with PBS and the lower 4 panels show animals injected with RAW cells expressing PTN/GFP. Cumulative results for the groups of mice monitored for 21 days post-surgery are shown graphically (FIG. 7B) as a ratio of blood flow in ischemic limb to that in the nonischemic limb at each time point (days).

### DESCRIPTION OF THE INVENTION

The invention is based on the inventors' surprising discovery that pleiotrophin (“PTN”) is related to neovascularization. It is believed that PTN is a plastogenic factor at least partially responsible for the transdifferentiation of macrophages and monocytes into endothelial cells. In fact, the inventors have surprisingly discovered that endothelial cells required for blood vessel formation need not originate from either pre-existing endothelial cells or their precursors; rather they may be generated de novo from other cell types. The various embodiments of the present invention set forth in greater detail, below, are predicated on this finding.

In one embodiment of the present invention, methods are provided to inhibit neovascularization by inhibiting the activity of PTN or its effects. This may be particularly advantageous in the treatment or prevention of diseases in which neovascularization is a component of disease pathology. Such diseases may include, by way of non-limiting example, cancer (e.g., tumor growth and metastasis), diabetic retinopathy and rheumatoid arthritis.

Conversely, in another embodiment of the present invention, methods are provided to promote neovascularization by enhancing/promoting the activity of PTN or its effects. This may be particularly advantageous in the treatment of diseases such as, but in no way limited to, ischemia and the development of ischemic tissue. Inhibition of PTN may also be advantageous in the treatment of vascular disease, because it is believed to play a role in plaque development through the endothelial cells that reside within the plaque structure.

There are many ways in which one may inhibit the activity or effects of PTN, which will be readily apparent to one of skill in the art. By way of example, one may administer a drug or peptide that blocks the action of PTN through its cell signaling cascade (e.g., by blocking PTN itself or by blocking its receptor). Alternatively, the action or effects of PTN may be inhibited with synthetic molecules, RNA-based technology or ribozymes. In one particular embodiment, one may employ interference RNA (“RNAi”) technology to inhibit the genetic activity of PTN.

There are also many ways in which one may upregulate, enhance or promote the activity of PTN. For example, one may employ any of a host of gene therapeutic techniques to cause or promote expression of the PTN gene in vivo. Such gene therapeutic approaches may be implemented with a vector, but this is not required. Alternatively, small molecules, peptides or other drugs that simulate the function of PTN may be administered in lieu of a promoter of PIN itself. Furthermore, by identifying the various components in the PTN cell signaling cascade, one may alternatively enhance that cascade by conventional methods, such as by administering a compound that is present in the cascade to increase signaling or by reducing the levels of a compound that stifles or otherwise reduces the effects of PTN through the cascade. In yet a further embodiment, one may administer an antibody against PTN and “immunize” an individual against its effects.

The delivery of any of the compounds or other products of the present invention may be performed locally or systemically, depending on the nature of the disease sought to be prevented or treated and other factors that will be readily apparent to one of ordinary skill in the art. In a still further embodiment of the present invention, PTN can be promoted or upregulated through non-gene cell therapy. In this embodiment, PTN may be overexpressed in, for example, a stem cell or a dendritic cell. The cell may then be implanted in a subject where it becomes part of the local tissue (e.g., blood vessel).

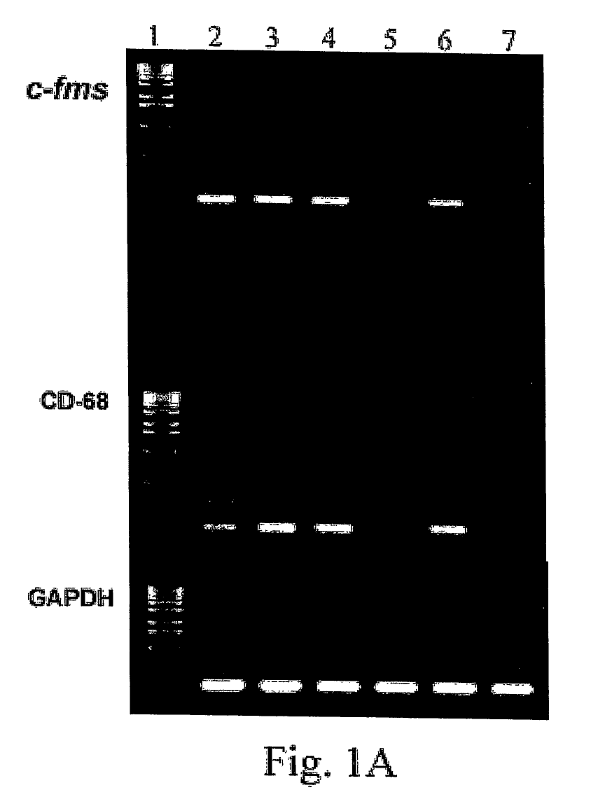

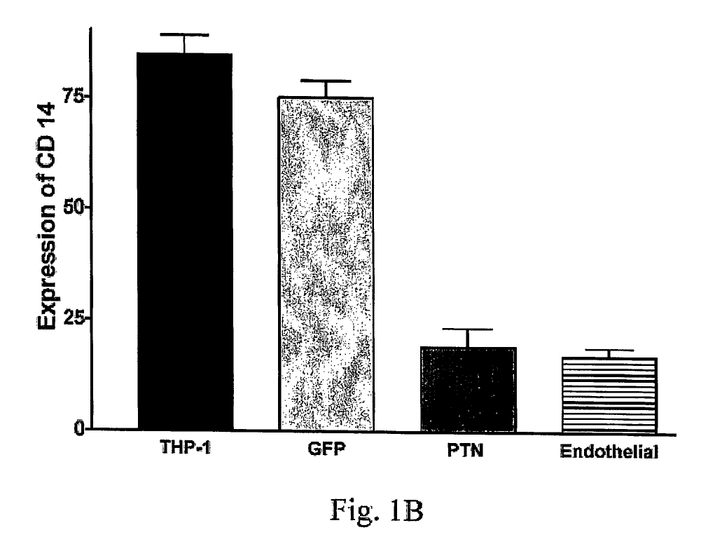

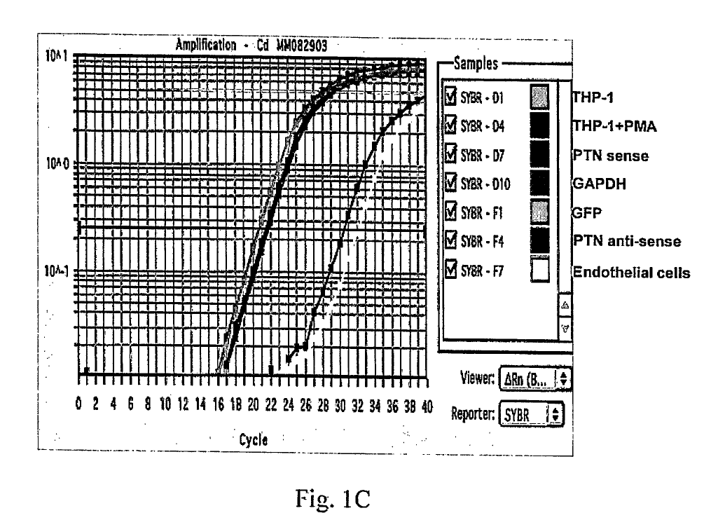

To remove the confounding variables that have plagued interpretation of transdifferentiation studies, clonal human THP-1 and mouse RAW monocytic cell lines were used to test the hypothesis that in the presence of PTN, monocytic cells transdifferentiate into endothelial-like cells and contribute to neovascularization of ischemic tissue. THP-1 and RAW cells were transduced with bicistronic retroviruses expressing PTN and green fluorescent protein (“GFP”), and the phenotype of monocytic cells was examined by semi quantitative RT-PCR. As expected, uninfected THP-1 cells (FIG. 1A, lane 2) or cells treated with PMA (lane 3) expressed monocytic cell markers, c-fms and CD68. Similarly, expression of GFP (lane 4) or PTN anti-sense strand (lane 6) in THP-1 cells had no effect on the expression of the monocyte/macrophage markers. In contrast, c-fms expression was undetectable and CD-68 expression was markedly down-regulated in THP-1 cells expressing PIN sense strand (lane 5). Negative control, human coronary artery endothelial cells did not express monocytic cell markers (lane 7). GAPDH amplification showed that the RT-PCR reactions proceeded efficiently for all tested samples.

To investigate this phenotypic modulation further, the inventors examined expression of CD14 by FACS. FACS analysis revealed that 85±4% of positive control THP-1 cells expressed CD14 (FIG. 1B, THP-1). Cells transduced with retroviral vector expressing GFP exhibited 75±4% CD14 positivity (FIG. 1B, GFP). In contrast, expression of CD14 was down-regulated (19±4%) in THP-1 cells transduced with retroviruses expressing PTN (FIG. 1B, PTN). This level of CD14 expression is similar (15±3%) to the expression level of CD14 in human coronary artery endothelial cells (FIG. 1B, Endothelial).

To validate the RT-PCR results, quantitative real-time PCR analysis was performed using primer sets specific for CD68. The data was obtained by comparing fold-induction normalized to the same gene from uninfected THP-41 RNA and with respective glyceraldehydes-3-phosphate dehydrogenase. The expression of CD68 was down-regulated approximately 6.4- to 7.6-fold in the PTN expressing THP-1 cells compared to THP-1 cells expressing anti-sense PTN or GFP, or uninfected THP-1 cells treated or untreated with PMA (FIG. 1C). The level of CD68 expression in PTN-expressing THP-1 is close to that of human endothelial cells (FIG. 1C). Similar results were obtained with real-time PCR analysis of c-fms (not shown). Collectively, the PCR data and flow cytometry results demonstrate that expression of PTN by monocytic cells leads to down-regulation of monocytic cell markers.

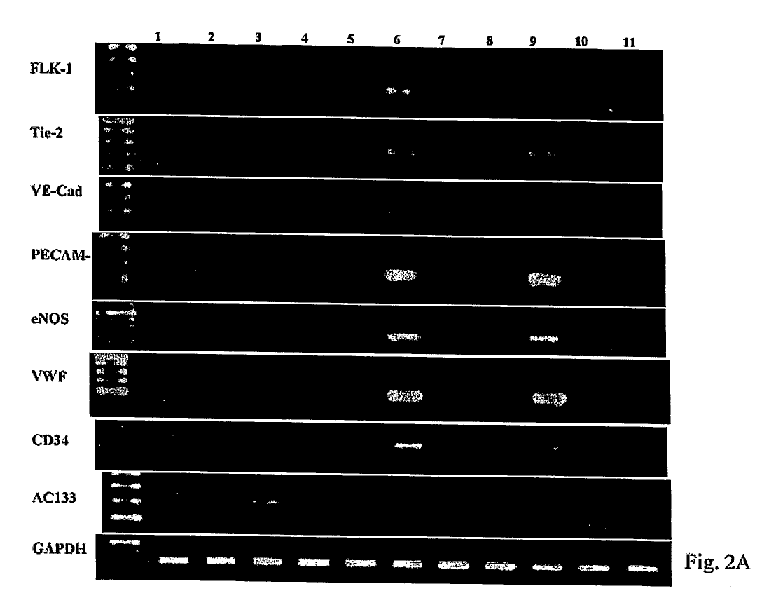

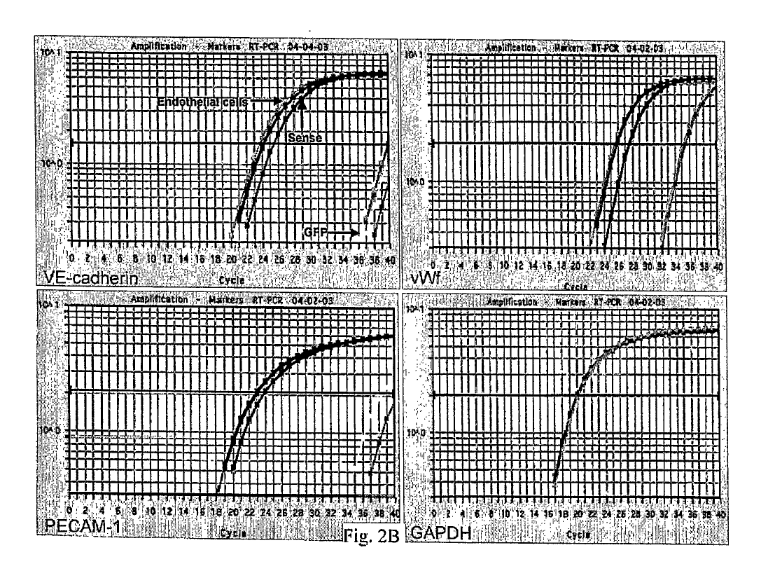

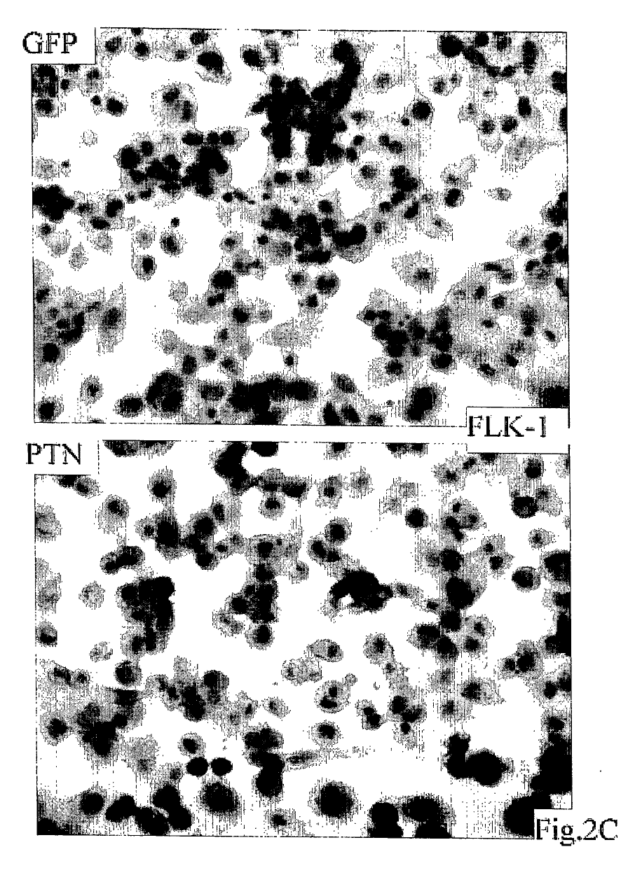

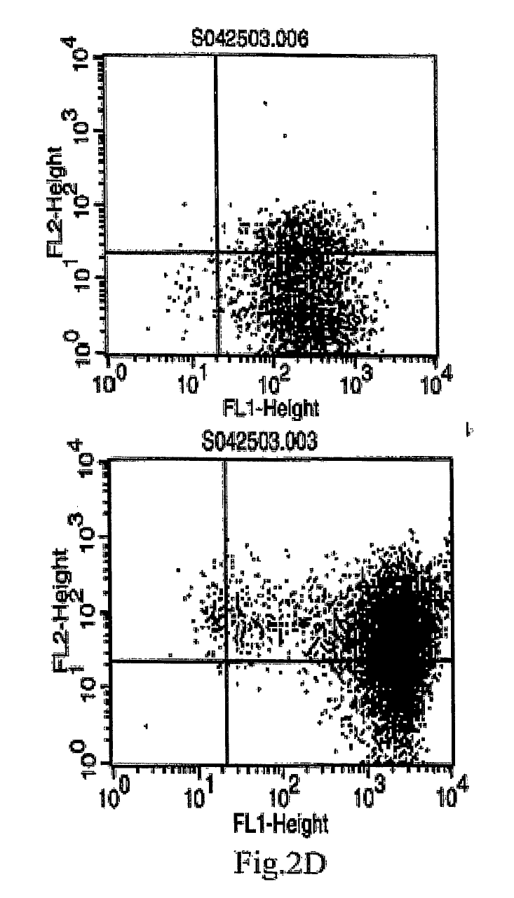

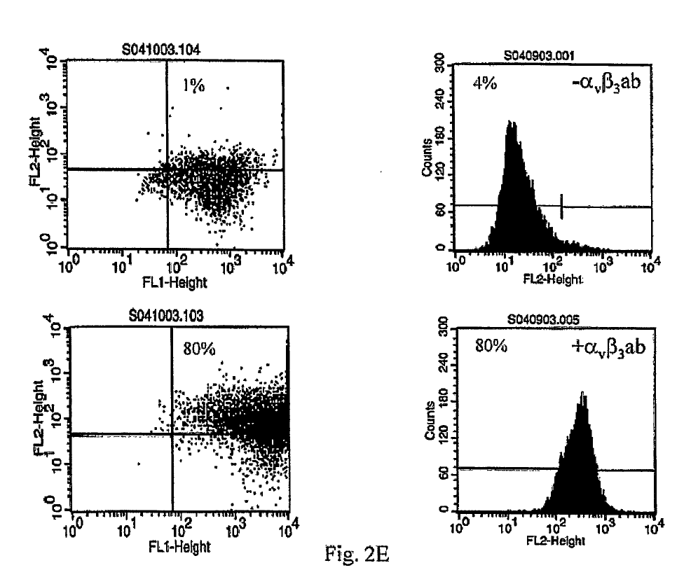

To evaluate whether transduced monocytic cells progressed to an endothelial-like phenotype, the inventors assessed them for the expression of endothelial cell markers by semi quantitative PCR. Mouse monocytic RAW cells (FIG. 2A, lane 1), human monocytic THP-1 cells (lane 2), and human promonocytic U937 cells (lane 3) did not express endothelial cell markers. However, THP-1 cells infected with PTN sense strand (lane 9) expressed vascular endothelial growth factor receptor-2 (“Flk-1”), Tie-2, vascular endothelial-cadherin (“VE-cad”), platelet endothelial cell adhesion molecule-1 (“PECAM-1”), endothelial nitric oxide synthase (“eNOS”), and the von Willebrand factor (“vWf”), similar to that of positive control human coronary artery endothelial cells (lane 6). In contrast, THP-1 cells infected with PTN anti-sense strand (lane 10) or the GFP control vector (lane 11) did not express endothelial cell markers. Likewise, endothelial cell markers were not detected in non-monocytic cells, such as NIH 3T3 cells (lane 4), human smooth muscle cells (lane 5), RPMI 8226 B lymphocyte plasmacytoma cell line (lane 7), and human skin fibroblasts (lane 8). The weak expression of FLK-1 in smooth muscle cells (lane 5) is consistent with the expression of this endothelial cell marker in human smooth muscle cells. See, e.g., A. Ishida et al., “Expression of vascular endothelial growth factor receptors in smooth muscle cells,” J. Cell. Physiol. 188, 359-68 (2001). In addition, RAW and THP-1 cells (uninfected and PTN-infected) did not express CD34 and CD133 markers. Collectively, these data demonstrate that expression of PTN leads to up-regulation of endothelial cell markers in THP-1 or RAW monocytic cells.

To confirm expression of endothelial markers in the monocytic cells, real-time PCR analysis was performed (FIG. 2B) for VE-cadherin, vWf, and PECAM-1. These data were normalized by comparison to a glyceraldehyde-3-phosphate dehydrogenase standard curve. The calculated gene expression levels (1.1×10⁵, 2.9×10⁵, and 0.7×10⁵ copies/100 ng RNA, respectively, P value all <0.001) are similar to the expression levels of these markers in the positive control human endothelial cells (1.5×10⁵, 3.2×10⁵, and 0.1×10⁵ copies/100 ng endothelial cell RNA, P value all <0.001). These data confirm semi-quantitative data shown in FIG. 2A.

Additional analyses of transdifferentiated cells were performed to document spatial distribution of endothelial cell markers. FLK-1 immunostaining showed that THP-1 cells infected with retrovirus expressing GFP did not stain with anti-FLK-1 antibody (FIG. 2C). In contrast, cells expressing PTN strongly stained with the antibody and the staining was concentrated in the membrane, in accordance with cellular distribution of FLK-1 (FIG. 2C). Additional staining revealed that VE-cadherin was expressed on the cell membrane while vWf expression was found to be dispersed throughout the cytoplasm (not shown). Further, the inventors determined the spatial distribution of Tie-2 in the THP-1 cells by flow cytometry. FACS analysis revealed that 72±5% of PTN-expressing THP-1 cells expressed Tie-2 (FIG. 2D, lower panel) compared to 7±3% of cells expressing GFP (FIG. 2D, top panel). Thus, the real-time PCR and flow cytometry confirmed reliability of transdifferentiation data obtained by semi-quantitative RT-PCR.

To determine the ability of the transdifferentiated cells to interact with extracellular matrix protein of blood vessels, the expression of αᵥβ₃ integrin was examined. FACS analysis revealed that 80±4% of THP-1 cells expressing PTN are positive for αᵥβ₃ (FIG. 2F, lower left panel), as compared to 1±3% of THP-1 cells expressing GFP (FIG. 2F, top left panel). The level of αᵥβ₃ integrin in the PTN-expressing THP-1 cells (80±4%) is similar to those of human coronary artery endothelial cells (FIG. 2F, lower right panel). Omission of anti-αᵥβ₃ antibody reduced positivity to 4±3% (FIG. 2F, top right panel).

Next, the inventors examined whether phenotypic modulation of monocytic cells by PTN is regulated at transcriptional levels. The expressions of many markers of endothelial cells are regulated by GATA-2 and GATA-3 zinc finger transcription factors. See, e.g., M. E. Lee et al., “Cloning of the GATA-binding protein that regulates endothelin-1 gene expression in endothelial cells,” J. Biol. Chem. 266, 16188-92 (1991); R. J. Gumina et al., “Characterization of the human platelet/endothelial cell adhesion molecule-1 promoter: identification of a GATA-2 binding element required for optimal transcriptional activity,” Blood 89, 1260-9 (1997); N. Jahroudi and D. C. Lynch, “Endothelial-cell-specific regulation of von Willebrand factor gene expression,” Mol. Cell Biol. 14, 999-1008 (1994); and P. J. Cowan et al., “The human ICAM-2 promoter is endothelial cell-specific in vitro and in vivo and contains critical Sp1 and GATA binding sites,” J. Biol. Chem. 273, 11737-44 (1998). Therefore, the expression of these factors was examined by semi-quantitative PCR. Monocytic RAW, U937, and THP-1 cells (FIG. 3A, lanes 1-3) as well as THP-1 cells infected with either PTN anti-sense strand (lane 10) or the GFP control vector (lane 11) did not express the transcription factors. Similarly, non-monocytic NIH 3T3 cells (lane 4), smooth muscle cells (lane 5), RPMI 8226 B lymphocyte plasmacytoma cells (lane 7), and human dermal fibroblasts (lane 8) did not express GATA-2 and GATA-3. In contrast, expression of PTN led to up-regulation of both GATA-2 and GATA-3 transcription factors in THP-1 cells (lane 9) at the levels comparable to those of control human endothelial cells (lane 6).

The monocytic cell lines that the inventors used are established cell lines with known monocytic cell characteristics that do not exhibit multipotent cell potentials. The results of RT-PCR studies described above support this notion as demonstrated by lack of expression of either hematopoietic stem cell markers (Tie-2) or endothelial progenitor markers (CD34). See, e.g., P. Carmeliet, “Angiogenesis in health and disease,” Nat. Med. 9, 653-60 (2003). In addition, RAW cells did not express PECAM-1, a definitive marker of mouse embryonic endothelial cells. See, e.g., A. Vecchi et al., “Monoclonal antibodies specific for endothelial cells of mouse blood vessels. Their application in the identification of adult and embryonic endothelium,” Eur. J. Cell Biol. 63, 247-54 (1994). Further, THP-1 cells did not express VEGF receptor FLK-1, a marker of undifferentiated, pluripotent endothelial cells. See, e.g., D. S. Kaufman et al., “Hematopoietic colony-forming cells derived from human embryonic stem cells,” Proc. Natl. Acad. Sci. USA 98, 10716-21 (2001); S. Levenberg et al., “Endothelial cells derived from human embryonic stem cells,” Proc. Natl. Acad. Sci. USA 99, 4391-6 (2002). Collectively, these data demonstrate that the monocytic cells do not exhibit characteristics of pluripotent cells. However, to further investigate the pluripotency potential of monocytic cells, the inventors determined expression of Oct-4, a marker for pluripotency. See, e.g., J. Nichols et al., “Formation of pluripotent stem cells in the mammalian embryo depends on the POU transcription factor Oct4,” Cell 95, 379-91 (1998); Y. I. Yeom et al., “Germline regulatory element of Oct-4 specific for the totipotent cycle of embryonal cells,” Development 122, 881-94 (1996). PCR analysis revealed that none of the monocytic cells examined expressed Oct-4 (FIG. 3A, lanes 1-3, 6, 9-11). Similarly, control non-monocytic cells such as smooth muscle cells (lane 5), endothelial cells (lane 6), and human fibroblasts (lane 8) did not express Oct-4. Interestingly, NIH 3T3 cells (lane 4) and RPMI 8226 B lymphocyte plasmacytoma cell line (lane 7) expressed Oct-4.

Additional analysis was performed to determine the relationship between distribution of transcription factors and endothelial cell marker by immunostaining and confocal microscopy. VE-cadherin expression was found to be localized on the surface of PTN-expressing RAW cells (FIG. 3B, red color) while GATA-2 expression was concentrated in the nucleus (FIG. 3B, blue color). The overlay showed the co-expression of the two endothelial cell markers. Such expression pattern was not detected in cells infected with the GFP control vector (not shown). Taken together, the PCR results and immunostaining data demonstrate that PTN has plastogenic activity, up-regulating endothelial cell phenotype in the THP-1 and RAW monocytic cells, possibly at the transcriptional level.

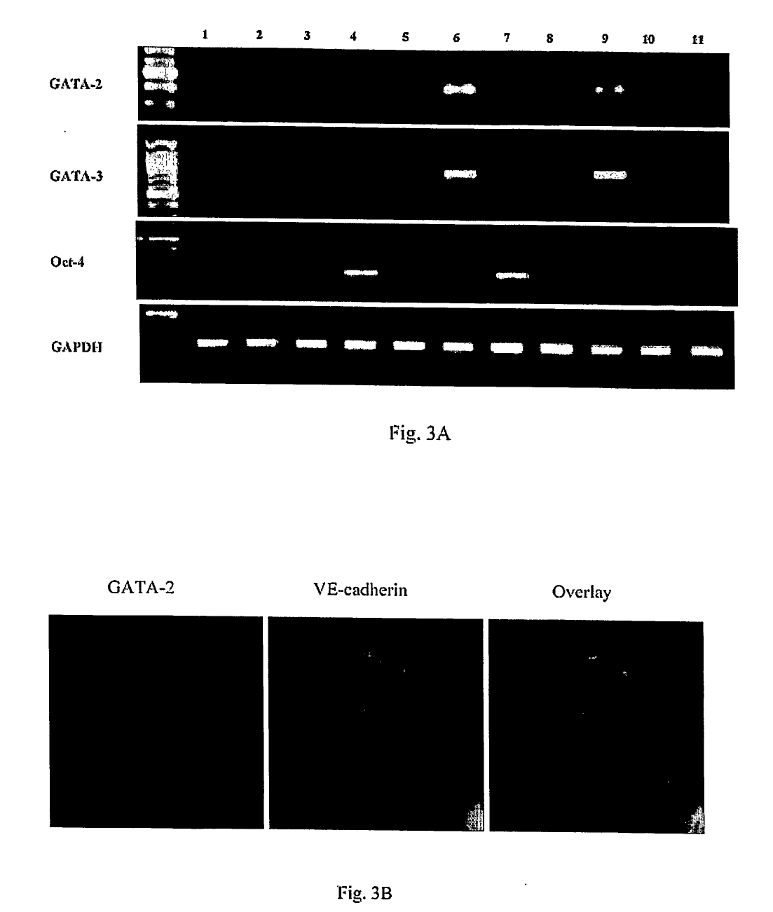

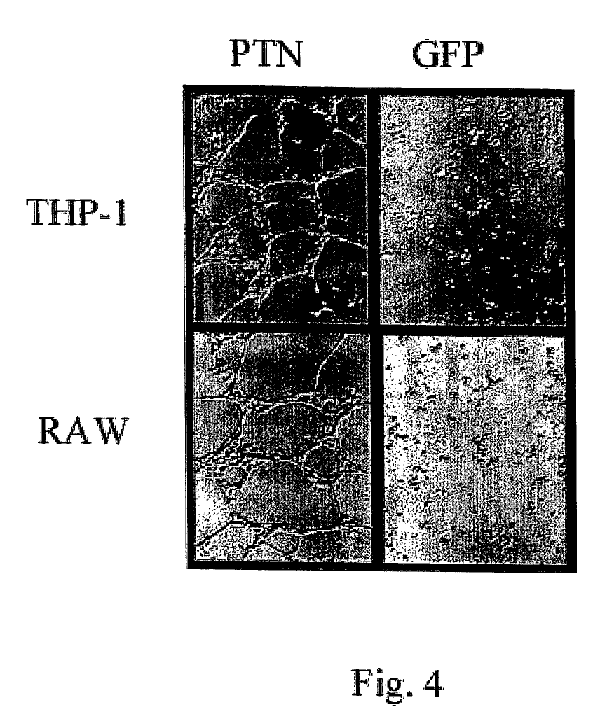

Finally, the inventors investigated the functional activity of transdifferentiated cells in vitro and in vivo. The ability of monocytic cells to form tubular structures in vitro was assessed by a three-dimensional fibrin matrix. Monocytic cells were cultured on fibrin gel and their morphology was monitored. The PTN-expressing THP-1 cells invaded the fibrin matrix and started to form tube-like structures in the three-dimensional gel as early as 4 days in culture and were observed up to 10-20 days. Afterwards, the structures became disorganized, forming multiple thin strands to thicker cord- and tubular-like structures (FIG. 4, PTN). These complex strands consisted of multiple rows of elongated cells in contact with each other. Cord formations continued to increase in complexity for up to 15-20 days. In contrast, THP-1 cells infected with the GFP control vector remained on top of the fibrin matrix and no network-like structures could be observed (FIG. 4, GFP). The positive control human coronary artery endothelial cells formed a tubular structure in the fibrin gel similar to THP-1 cells infected with PTN sense strand (not shown). Similar results were obtained with RAW cells (not shown). These data demonstrate that PTN expression confers the ability to THP-1 and RAW cells to rearrange in the fibrin gel with extended cytoplasm and interact with surrounding cells, similar to endothelial cells.

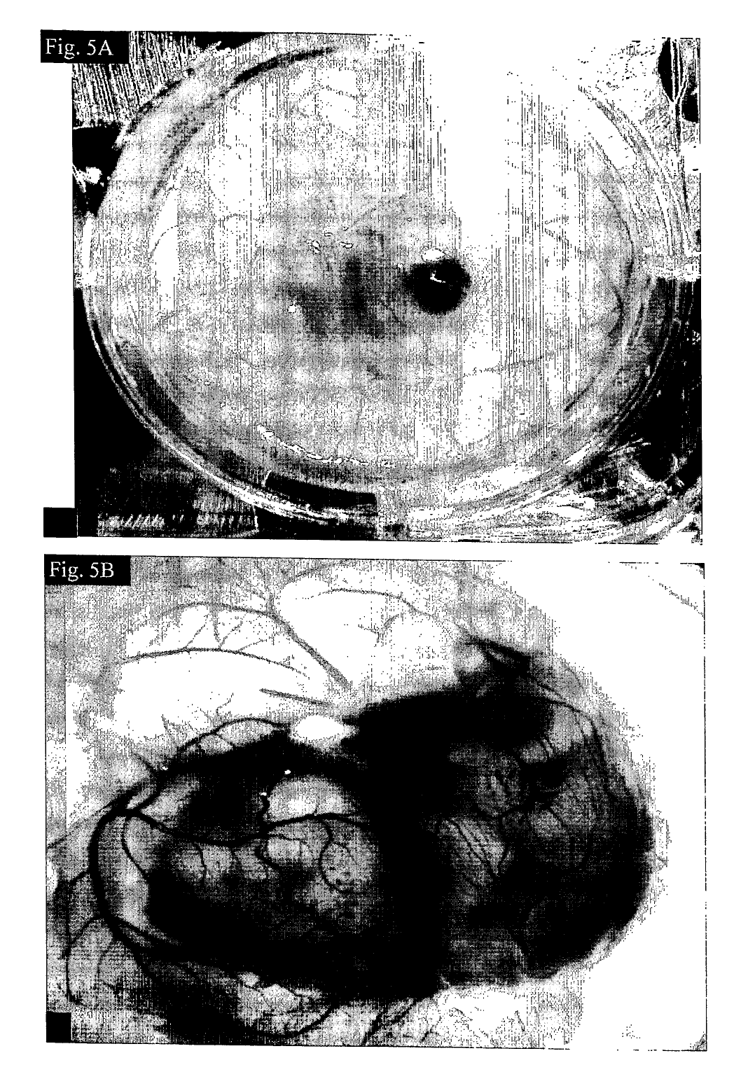

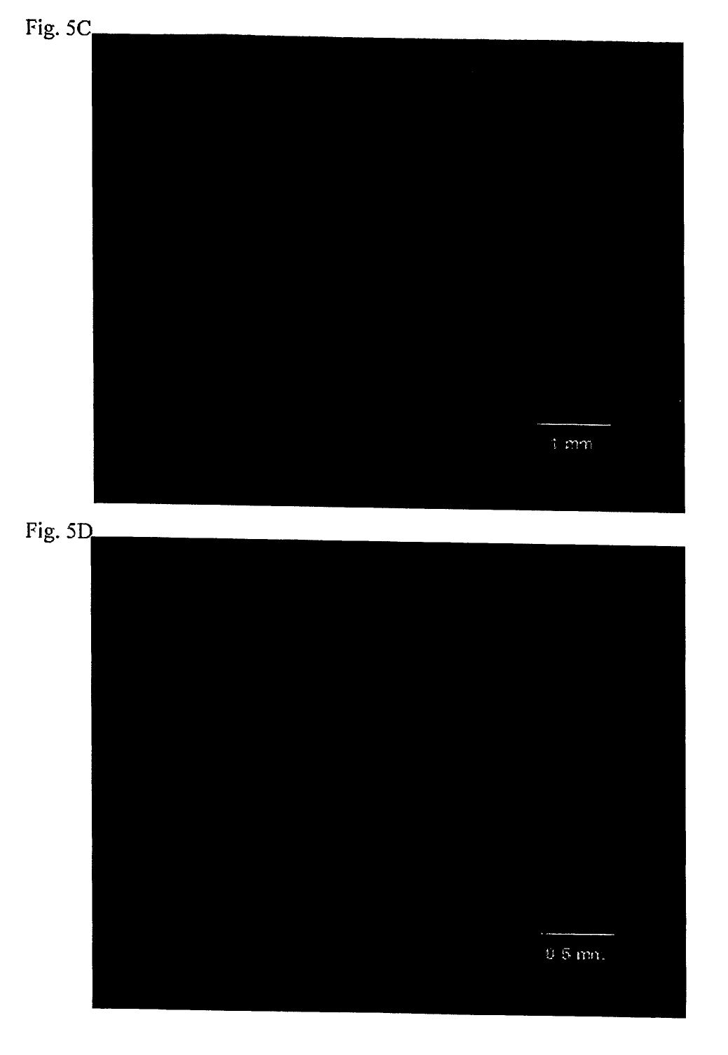

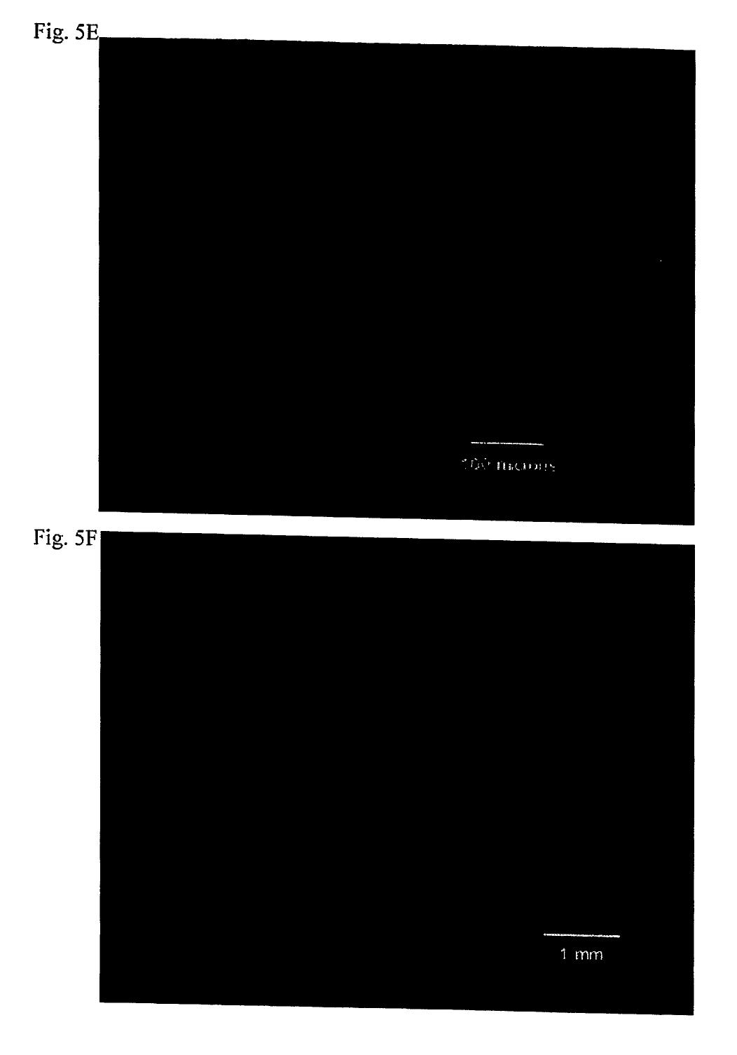

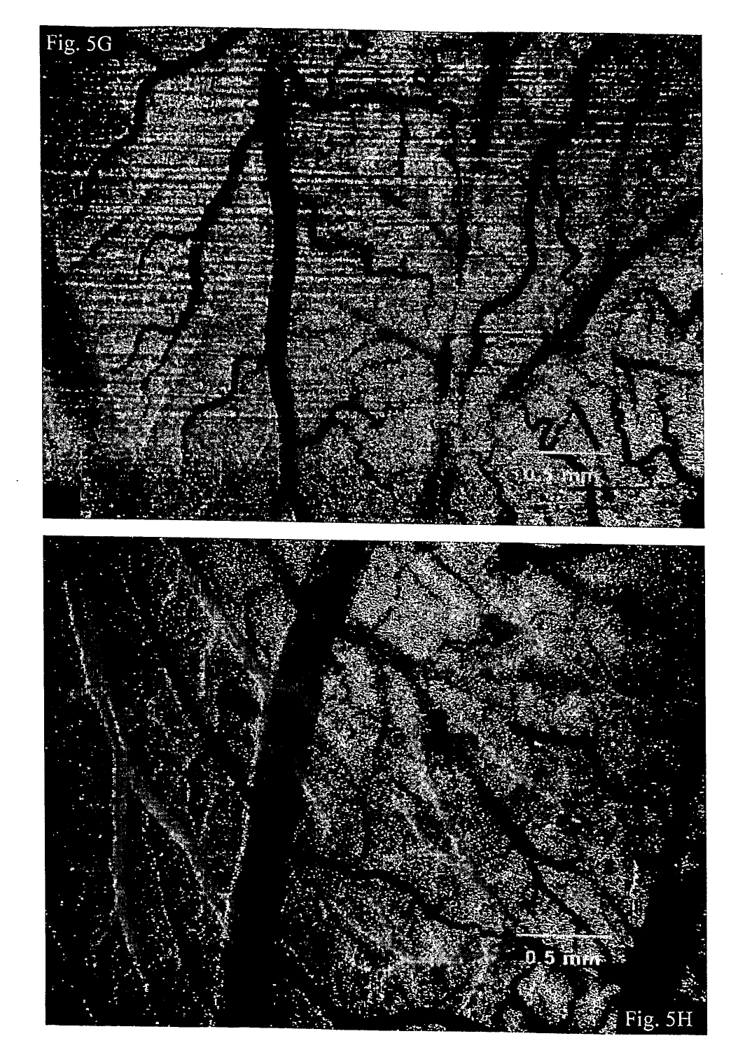

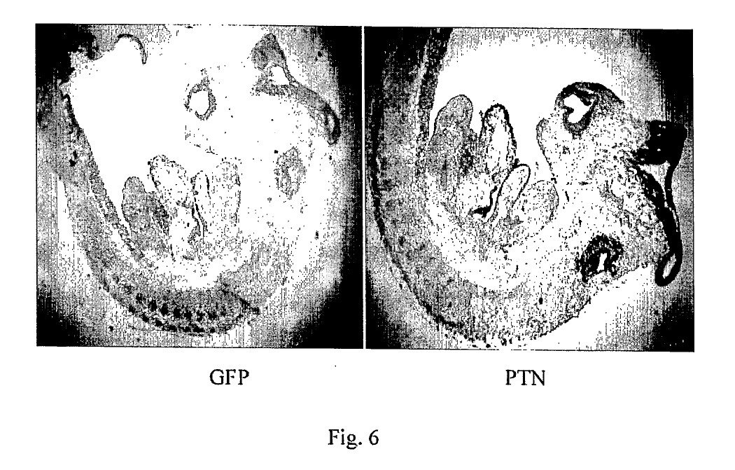

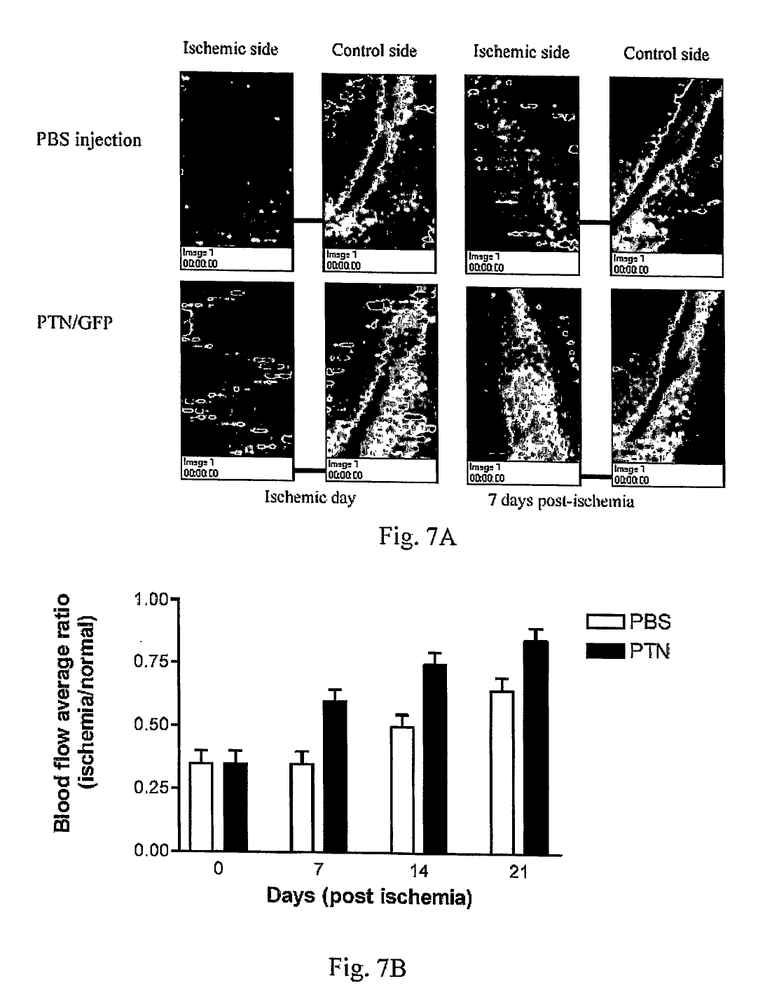

To determine whether transdifferentiated cells contribute to neovascularization in vivo, the inventors used quail chorioallantotic membrane (CAM), chicken-mouse chimera, and murine hindlimb ischemia assays. Fertilized eggs of Japanese quail were cultured ex-ovo. See, e.g., P. Parsons-Wingerter et al., “A novel assay of angiogenesis in the quail chorioallantoic membrane: stimulation by bFGF and inhibition by angiostatin according to fractal dimension and grid intersection,” Microvasc. Res. 55, 201-14 (1998). RAW cells expressing PTN were then transplanted onto CAM of the E7 embryos (FIG. 5A, panel a). RAW cells or 293 cells expressing GFP were used as controls in addition to PBS. After 3 days, at E10 (FIG. 5A, panel b), CAMs were fixed, dissected out, mounted onto a glass slide, and the images were analyzed by fluorescence and confocal microscopy. Implantation of RAW cells expressing both PTN/GFP led to their integration into newly developed quail vascular tree in 9/12 embryos [FIG. 5A, panel c (2× magnification); panel d (10× magnification); panel e (20× magnification)]. In addition, FIG. 5A shows that PTN-expressing cells became incorporated into large and small blood vessels at various branches. Similar results were obtained by implantation of PTN-expressing THP-1 cells (not shown). In contrast, no RAW cells expressing GFP were incorporated into the newly developing blood vessels in 8/10 embryos examined (FIG. 5A, panel f). Furthermore, no fluorescence was detected when either 293 cells or PBS was added to the embryos (not shown).

The confocal image of the newly formed fluorescent-labeled blood vessels were overlapped with differential interference contrast image (“DIC”) to improve the contrast in order to observe incorporation of GFP-labeled cells into the blood vessels in the context of full view of developing vascular tree. The DIC images showed that monocytic cells expressing GFP did not incorporate into blood vessels (FIG. 5A, panel g). In contrast, PTN-expressing RAW cells integrated into some of the newly developed blood vessel (FIG. 5A, panel h), generating chimeric quail-mouse blood vessels. Taken together, the confocal images clearly show that the transdifferentiated monocytic cells have all the necessary biological information to home in and become integrated into the developing blood vessels.

The ability of transdifferentiated cells to incorporate into vasculature is further underscored by their capacity to integrate into developing blood vessels of multiple organs. RAW cells expressing PTN (1×10⁶) were injected intracardially into stage 16-17 chick embryos and the fate of these cells was determine 2-3 days post-injection by anti-GFP antibody. Most of the resulting immunopositivity appeared along the vessels in the head, eyes, and intersomitic regions (FIG. 5B, PTN panel), and forming a network structure in the embryo injected with RAW cells expressing PIN/GFP. In contrast, embryos injected with RAW cells expressing GFP did not stain (FIG. 5B, GFP panel). Taken together with CAM data, and while not wishing to be bound by any particular theory, the inventors concluded that PTN-expressing RAW cells, but not GFP-expressing cells, have the fundamental biological information that allow them to integrate into newly developed blood vessels in different organs.

Past studies have shown that PTN mRNA is up-regulated in macrophages found in the developing microvasculature after acute ischemic brain injury. See, e.g., A. Takeda et al., “Induction of heparin-binding growth-associated molecule expression in reactive astrocytes following hippocampal neuronal injury,” Neuroscience 68, 57-64 (1995); H. J. Yeh et al., “Upregulation of pleiotrophin gene expression in developing microvasculature, macrophages, and astrocytes after acute ischemic brain injury,” J. Neurosci. 18, 3699-707 (1998). This suggests a role for macrophage-derived PTN in neovascularization of ischemic tissues. To provide functional evidence for transdifferentiated monocytic RAW cells in perfusion of ischemic tissue, mild hindlimb ischemia were induced in BALB/C mice, according to methods described in T. Couffinhal et al., “Mouse model of angiogenesis,” Am. J. Pathol. 152, 1667-1679 (1998); and blood flow was monitored by laser Doppler perfusion imaging at days 7, 14, and 21 post-surgery. BALB/C background mice were selected for this experiment because RAW cells are congenic to this mouse strain and thus avoiding a potential graft-versus-host complication. One day after creating unilateral hindlimb ischemia, 5×10⁵ RAW cells expressing PTN were injected into the tail vein. Injection of PBS was used as a control. Blood flow was measured by laser Doppler perfusion imaging before and various times after surgery. A representative laser Doppler perfusion image of mice injected with RAW cells expressing PTN at day 7 is shown in FIG. 5C. Hindlimb blood flow recovery in mice receiving PTN-expressing RAW cells was significantly higher than that of control mice. In comparison to the reference limb, BALB/C mice injected with PTN-expressing RAW cells had 70±3% increase in blood flow at 7 days, 50±4% at 14 days and 30±3% at 21 days compared to control animals injected with PBS at similar time points (FIG. 5C).

In summary, the inventors used several experimental approaches to test the hypothesis that monocytic cells have the potential to transdifferentiate into endothelial cells. The inventors' findings suggest that expression of PTN by monocytic cells has an autocrine impact on the cells leading to down-regulation of monocytic cell markers and up-regulation of endothelial cell characteristics. Cultured cells expressing PTN, but not control cells, formed tube-like structure in fibrin gel. In vivo functional studies revealed that the PTN-expressing monocytes hone to and become integrated into newly developing blood vessels. In addition, transdifferentiated monocytes promote perfusion of ischemic tissues. Because monocytes/macrophages have the ability to fuse spontaneously with other cell types and adopt their phenotype, using homogeneous cells allowed us to demonstrate that the transdifferentiation of monocytes into endothelial cells is not mediated by cell fusion. See, e.g, N. Terada et al., “Bone marrow cells adopt the phenotype of other cells by spontaneous cell fusion,” Nature 416, 542-5 (2002). Taken together, these findings suggest that the role of monocyte in neovascularization is both direct (transdifferentiation into endothelial cells) and indirect (releasing growth factors and cytokines).

The inventors found that monocyte transdifferentiation is fully dependent on PTN expression, suggesting that the gene rather than the cell regulates the phenotypic conversion; thus identifying a new plastogenic role for PTN gene. Past studies have shown that PTN stimulates proliferation of peripheral blood-derived monocytes and tissue macrophages, suggesting that PTN is capable of promoting neovascularization through two different mechanisms: 1) stimulating proliferation of residing monocytes/macrophages that release wide array of angiogenic factors and cytokines, and 2) transdifferentiation of monocytes into endothelial cells. Prior studies have also shown that tumor cells can assume endothelial-like cell characteristics. For example, expression of VEGF, Flk-1, and Flt-1 has been detected in a variety of human tumor cells of nonendothelial origin. See, e.g., R. Masood et al., “Vascular endothelial growth factor (VEGF) is an autocrine growth factor for VEGF receptor-positive human tumors,” Blood 98, 1904-13 (2001). These included melanoma, ovarian, pancreatic, prostate carcinomas, and breast cancer. While there is no information about the mechanism that contributes to this phenotypic modulation of tumor cells, it is important to note that there is a good correlation between PTN synthesis and expression of endothelial cell markers in these tumor cells; for instance, a high level of PTN expression has been detected in melanoma cells, pancreatic cancer, prostate cancer, and breast tumor. See, e.g., F. Czubayko et al., “Melanoma angiogenesis and metastasis modulated by ribozyme targeting of the secreted growth factor pleiotrophin,” Proc. Natl. Acad. Sci. USA 93, 14753-14758 (1996); D. Weber et al., “Pleiotrophin can be rate-limiting for pancreatic cancer cell growth,” Cancer Res. 60, 5284-8 (2000); F. Vacherot et al., “Involvement of heparin affin regulatory peptide in human prostate cancer,” Prostate 38, 126-36 (1999); and A. T. Riegel and A. Wellstein, “The potential role of the heparin-binding growth factor pleiotrophin in breast cancer,” Breast Cancer Res. Treat. 31, 309-14 (1994). In light of the inventors' findings, it is tempting to suggest that PTN may be in part responsible for the up-regulation of endothelial cell markers in these tumors.

Collectively, the inventors' PTN-mediated transdifferentiation data identifies a novel neovascularization paradigm, directly linking inflammation to neovascularization and suggests a new source for endothelial cells during neovascularization, in addition to the two known sources of endothelial cells (i.e., angiogenesis and endothelial progenitor cells). These results are believed to identify “angiogenic switch(s)” of potential broad importance in human tumors and inflammatory diseases, advance knowledge of vasculogenesis, and support the identifications of sites for therapeutic intervention.

### EXAMPLES

### Cloning of Full-Length Human PTN

The inventors used the full-length human PTN open reading frame (accession # NM_{—}002825) to clone the full-length cDNA using PCR and specific primers for PTN [5'AAAATGCAGGCTCAACAGT (SEQ ID NO: 1) and 5'TGTTTGCTGATGTCCTTT (SEQ ID NO: 2)]. The veracity of the cloned product was verified by DNA sequencing.

### Generation of Stably Transduced THP-1 and RAW Cells Using Retrovirus

Bicistronic retroviral vectors were constructed from the pLP-EGFP-C1 retroviral expression system (obtained from Clontech). The pLP-EGFP-C1 plasmid containing the enhanced GFP gene under the control of CMV promoter was modified by adding an internal ribosomal entry site (“IRES”) sequence after the GFP segment. The full-length cDNA of human PTN was cloned into the BamH/Notl sites of pLP-C1-IRES-GFP. The retrovirus was packaged using a 293 packaging cell line (obtained from Clontech). After transfection of packaging cells, viruses were collected, and retroviral titers between 1×10⁶ and 2×10⁷ cfu/ml were determined by limiting dilution with NIH3T3 cells. For infection, 4×10⁵ THP-1 or RAW cells were plated in 25-cm² flasks 24 h before infection in normal growth medium (RPMI/10% FBS) to obtain exponentially growing cultures. The cells were transfected (approximate MOI 2.5-25 cfu/cell) using standard protocols. Polyclonal populations of cells (3×10⁵ each) expressing low or high levels of the EGFP reporter gene were then isolated by flow cytometry. Immediate post-sort analysis confirmed the isolation of distinct populations of cells based on GFP expression (not shown). Cells were subcultured for an additional passage and analyzed for expression of the GFP reporter gene by flow cytometry and for expression of PTN by Western blot analysis.

### ELISA Assay

To measure the level of secreted PTN by the transduced cells, media collected from THP-1 cells 24, 48, and 72 h after plating and the concentration of PTN was measured by standard ELISA assay. Media collected from THP-1 cells treated with 25 ng/ml TNF-α were used as a positive control. The concentration of PTN increased with time (5±2 ng/ml, at 48 h and 12±4 ng/ml at 72 hr) slightly lower than PTN levels induced by TNF-α at the indicated times (10±3 ng/ml and 20±4 ng/ml).

### RT-PCR Analysis

Reverse transcription (RT)-PCR conditions were as follows: RT and activation: 30° C. for 30 min, 95° C. for 15 min; amplification: 94° C. for 1 min, 55° C. for 1 min, 72° C. for 1 min; and final extension: 72° C. for 10 min.

Optimal cell cycle numbers were determined for each gene to ensure that conditions were in the linear range of PCR amplification. They are illustrated below in Table 1, along with an indication of the primer sequences.

TABLE 1
Optimal Cycle Numbers and PCR Primer Sequences

| | | |
|---|---|---|
| Gene | Number Cycles | Primer Sequences |

| | | |
|---|---|---|
| PECAM-1 | 28 | GCTGTTGGTGGAAGGAGTGC |
| | | GAAGTTGGCTGGAGGTGCTC |
| | | [SEQ ID NOs: 3 & 4] |
| VE-cad | 32 | CCGGCGCCAAAAGAGAGA |
| | | CTGGTTTTCCTTCAGCTGGAAGTGGT |
| | | [SEQ ID NOs: 5 & 6] |
| CD34 | 30 | TGAAGCCTAGCCTGTCACCT |
| | | CGCACAGCTGGAGGTCTTAT |
| | | [SEQ ID NOs: 7 & 8] |
| Flk-1 | 31 | CAACAAAGCGGAGAGGAG |
| | | ATGACGATGGACAAGTACCC |
| | | [SEQ ID NOs: 9 & 10] |
| Tie-2 | 31 | CCTTAGTGACATTCTTCC |
| | | GCAAAAATGTCCACCTGG |
| | | [SEQ ID NOs: 11 & 12] |
| eNOS | 37 | ACTTCTGCGCCTTTGCTC |
| | | TGTCCAGGAAGAAGGGGTGAGA |
| | | [SEQ ID NOs: 13 & 14] |
| VWF | 32 | GGAAGACCCAGTGCTGTGAT |
| | | GTCTTCCTGCACTCCAGCTT |
| | | [SEQ ID NOs: 15 & 16] |
| GATA-2 | 33 | CCCTAAGCAGCGCAGCAAGAC |
| | | TGACTTCTCCTGCATGCACT |
| | | [SEQ ID NOs: 17 & 18] |
| GATA-3 | 29 | ACCCCACTGTGGCGGCGAGAT |
| | | CACAGCACTAGAGACC |
| | | [SEQ ID NOs: 19 & 20] |
| OCT-4 | 31 | GAGAACAATGAGAACCTTCAGGAGA |
| | | TTCTGGCGCCGGTTACAGAACCA |
| | | [SEQ ID NOs: 21 & 22] |
| GAPDH | 24 | AGCCACATCGCTCAGACACC |
| | | GTACTCAGCGGCCAGCATCG |
| | | [SEQ ID NOs: 23 & 24] |

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

What is claimed is:

1. A method of transdifferentiating a monocytic cell into an endothelial cell, comprising:
providing a monocytic cell; and
artificially increasing the expression of PTN in the monocytic cell such that the monocytic cell transdifferentiates into an endothelial cell.

2. The method of claim 1, wherein artificially increasing the expression of PTN in the monocytic cell further comprises:
transducing the monocytic cell with a retrovirus expressing PTN.

3. The method of claim 2, wherein the retrovirus is a bicistronic retrovirus.

4. The method of claim 1, wherein the monocytic cell is a RAW cell or a THP-1 cell.

5. An endothelial cell, produced by the method, comprising:
providing a monocytic cell; and
artificially increasing the expression of PTN in the monocytic cell such that the monocytic cell transdifferentiates into an endothelial cell.

6. The endothelial cell of claim 5, wherein artificially increasing the expression of PTN in the monocytic cell further comprises:
transducing the monocytic cell with a retrovirus expressing PTN.

7. The endothelial cell of claim 6, wherein the retrovirus is a bicistronic retrovirus.

8. The endothelial cell of claim 5, wherein the monocytic cell is a RAW cell or a THP-1 cell.

9. A method of inhibiting neovascularization, comprising:
providing a compound that inhibits the biological activity of pleiotrophin (PTN); and
administering the compound to a mammal in a therapeutically effective quantity such that neovascularization is inhibited.

10. A method of promoting neovascularization, comprising:
providing a compound that enhances or promotes the biological activity of pleiotrophin (PTN); and
administering the compound to a mammal in a therapeutically effective quantity such that neovascularization is promoted.
